(19) United States
(12) Patent Application Publication
(10) Pub. No.: US 2011/0004065 A2
(43) Pub. Date: Jan. 6, 2011

(54) INTUBATION TUBE

(75) Inventor: Christoph SCHRAMM, Besigheim (DE)

Correspondence Address: OBLON, SPIVAK, MCCLELLAND MAIER & NEUSTADT, L.L.P., 1940 DUKE STREET, ALEXANDRIA, VA, 22314, UNITED STATES, 703-413-3000, 703-413-2220, patentdocket@oblon.com

(73) Assignee: RUPRECHT-KARLS-UNIVERSITAET HEIDELBERG, Heidelberg (DE)

(21) Appl. No.: 12/518,161

(22) PCT Filed: Dec. 3, 2007
(86) PCT No.: PCT/DE07/02172

§ 371 Date: Aug. 20, 2009

(30) Foreign Application Priority Data
Dec. 6, 2006 (DE) 10 2006 057 809.0

(51) Int. Cl. A61B 1/06; A61M 16/00

(52) U.S. Cl. 600/120; 128/200.26; 600/249

(57) ABSTRACT

An interventional aid for medical interventions, including a tube body with a tube jacket which permits intubation and which, while leaving a lumen to permit ventilation, includes a functional aid for performing an interventional function. The functional aid includes an orientation mechanism orienting the tube body during the medical intervention.

The present invention concerns what is claimed in the preamble and therefore relates to interventional aids for medical interventions.

There are a great many medical interventions in which a patient's oxygen supply is put at risk or at least impaired. This may be the case when the patient, for example under anesthesia, is no longer able to breathe spontaneously. Another situation that often arises is one in which, for examination purposes or treatment purposes, devices have to be inserted into the body through the trachea and as far as the lung. The maximum possible duration of such interventions is strictly limited by the need to continue supplying the patient with oxygen.

It is known and customary to intubate the patient in a large number of situations in which it is necessary to ensure ventilation. In intubation, an endotracheal tube is inserted by means of intubation, which typically takes place through the throat, but can sometimes also be done through the nose.

Intubation itself is a critical procedure. It must be ensured that the endotracheal tube is inserted sufficiently deeply and within a sufficiently short time, which, in adults, requires pushing the tube past the vocal cords. While experienced physicians are generally able to do this without any problems, complications may sometimes arise, for example on account of special anatomical circumstances, or in emergency cases in which the nasopharyngeal spaces are not accessible, or are only accessible to a limited extent, because they are closed, destroyed or the like.

A particular problem is that of being able to position the tube quickly and precisely. Aids are already known for this purpose, for example the laryngoscope, but also other aids, such as endoscopes insertable into the lumen of the endotracheal tube, which have to be removed to permit ventilation after the endotracheal tube has been fitted.

It is also already known to provide an endotracheal camera. For this purpose, US 2005/0177024 A1 proposes a camera and a radiofrequency transmitter that are coupled to an endotracheal tube in order to acquire an image of tissue at the proximal end of the endotracheal tube in real time. The image recorded by the camera is transferred to an inexpensive radiofrequency receiver situated close by and sent to a video monitor in order to display the image. The use of a wireless transmission system is intended to avoid the presence of wires and cords that could otherwise catch in each other and could have the effect of the endotracheal tube being accidentally withdrawn from the patient or repositioned in the patient. However, the positioning is also critical in the known arrangement.

In view of the great danger to which the patient is exposed by complications during ventilation, it is desirable here to permit improvements in the positioning of the endotracheal tube.

Further problems arise for patients if, in addition to purely permitting ventilation, further requirements need to be satisfied, for example regular monitoring of the airways of injured patients in order to ensure that liquids can be aspirated sufficiently quickly during an operation, the removal of foreign bodies from airways, and long-term monitoring, for example in intensive care units, of comatose patients or of patients being deliberately kept in a coma, etc. In minimally invasive interventions specifically, a problem is that the intervention time is determined by the maximum permissible time for which respiration can be limited.

It is desirable to at least partially remedy at least some of the problems discussed. It is true that devices are already known for minimally invasive interventions. Some examples of patents that deal with intubation and endoscopy include:

DE 195 18 148 A1, DE 690 23 930 T2, U.S. Pat. No. 5,803,080, DE 10 2004 026 619 A1, DE 698 30 051 T2, EP 1 177 809 A1, US Patent 2005/0177024. However, the instruments shown in these documents prove inadequate in practice.

The object of the present invention is to make available something that is novel and industrially applicable.

This object is achieved by what is claimed in the independent claims.

Preferred embodiments are set forth in the dependent claims.

The present invention thus proposes, in a first basic concept, an interventional aid for medical interventions, comprising a tube body with a tube jacket which permits intubation and which, while leaving a lumen to permit ventilation, is provided with a functional aid for performing an interventional function, wherein the functional aid is designed with an orientation means for orienting the tube body during the medical intervention.

Thus, with the invention, a particularly simple positioning of an endotracheal tube is first proposed which, even in cases where an experienced physician encounters problems that make positioning of the endotracheal tube difficult, permits safe and rapid positioning of the tube without any risk of permanent damage to the patient, simply by the tube body being provided with orientation means for orienting the proximal end of the tube body during the intubation procedure.

It was recognized that, by means of the described wall integration, such orientation means or steering means can also be provided in an endotracheal tube without, as is customary in the prior art, having to insert a steerable endoscope into the endotracheal tube that is to be positioned or oriented, with the tube body of the endotracheal tube then being moved by means of the movement of the endoscope. It will be noted that the walls of the endotracheal tube can be made so thin, without impairing their function, that, on the one hand, a lumen permitting ventilation remains along the entire length and, on the other hand, the remaining wall thickness, although already limited in overall circumference for typical anatomies, is still sufficient for the orientation means and/or steering means to be provided therein and/or thereon. The wall also proves to be sufficiently stable at those places or near those places where forces are to be transferred distally, that is to say from outside the patient, for example by the physician or medical staff performing the intubation or examination or intervention, when tensioning means or steering means are embedded in the wall and engage at the proximal end of the tube or near this. One of the reasons for this is obviously the fact that, after the start of the positioning procedure, a stabilization is already provided by the lateral body parts, i.e. the trachea or bronchi and the like, and safely prevents kinking of the tube body during the intubation or during the medical intervention. Use is also made of the fact that, because the tube jacket itself is provided with the functional aid, the complication feared in the prior art and concerning displacement when the wires are pulled does not arise.

In the preferred variant, the interventional aid is designed for at least one medical intervention from the group comprising bronchial lavage, bronchoscopy, biopsy, anesthesia, intensive care, intubation, aspiration, ENT surgery, thoracic surgery and/or radiotherapy, in particular for placement of radioactive elements or for afterloading, and/or for training in any one of the aforementioned interventions. It will be appreciated that designing the interventional aid for training makes it possible, for example, to omit sterilization, sterile handling and the like and, if appropriate, additional sensors can be provided purely for monitoring the success of the training, which can if appropriate also take place with a reduction in size of the otherwise necessary lumen. Moreover, for bronchial lavage for example, a flushing possibility is provided, that is to say the tube jacket or the inside of the body can be additionally provided with a hose for flushing or, if separate hoses are used, for delivery and removal of liquid with two hose lines.

For the purpose of bronchoscopy, viewing means can be inserted through the lumen, which requires that the lumen be dimensioned with a size that permits ventilation even with the viewing means, for example an endoscope.

However, it is alternatively possible, and clearly preferable, to provide conducting means for transmitting images from the inside of the body, which conducting means are fixedly connected to the wall or a wall (if appropriate an inside wall or partition wall) of the tube body. Such conducting means for transmission of images may be highly expedient in pure bronchoscopy but are also of advantage to the anesthetist when intubating in difficult situations for and, in intensive care too, they facilitate the monitoring of the status of the patient. This may be expedient, for example, when internal hemorrhaging is feared, or during operations in which, although the lung is not being operated on, the patient nevertheless has to be moved on the operating table, as a result of which there must be a fear of the already intubated tube being displaced. In such a case, an orientation of the tube can be newly optimized by moving the endotracheal tube and at the same time observing it.

The interventional aid not only facilitates known interventions, such as intubation or bronchoscopy, but also permits new designs of operations, in particular using a minimally invasive approach. It is easily possible, by virtue of its steerability, for the interventional aid to be advanced, preferably with visual monitoring, deep into the bronchi, for example as far as the segmental bronchi, where there are strong ramifications, and to do so without the need for a straight line of view along which an operating surgeon is able to observe the site, and without the ventilation, as in the prior art, being impeded in order to permit observation of the positioning.

The design for the minimally invasive intervention requires, in addition to a lumen size that permits the use of the minimally invasive surgical aids such as forceps, pincers, scalpels, etc., without impeding the ventilation, also the design of the interventional aid in a suitable length, such that the tube can be advanced as far as the desired site.

If, as is desirable, a cuff is provided with which the tube is sealed off near the larynx, as is customary in intubation of adults, the cuff of an interventional aid designed for interventions in the segmental bronchi will accordingly be arranged near the distal end. In other words, the interventional aid is possibly, but not necessarily, designed for an intervention deep in the inside of the body by moving the cuff away from the proximal end toward the distal end. It must be reckoned that interventional aids with different cuff position will be necessary for different interventions.

In cases in which the interventional aid is advanced to a deep position, it will also be preferable to provide openings under the cuff, to ensure that ventilation takes place not only of those lung areas that can be supplied with air or oxygen, anesthetic gas, etc., from the lumen opening at the proximal end, but also of the entire lung via the side opening. It will be noted that each one of the listed features, such as the widening of the lumen to permit the insertion of surgical interventional aids, the cuff displacement, and the provision of side openings, can individually already facilitate and/or permit minimally invasive interventions, but that the design for minimally invasive operations does not necessarily have to be made dependent on the presence of such features.

It will further be noted that, even when side openings are present for ventilation of lung areas not reached by the tube, the wall thickness is not impaired such as to prevent the stability of the interventional aid and/or intubation. Instead, the increased flexibility of the tube in the presence of ventilation holes will make positioning even easier to perform, which is advantageous specifically as regards the required deeper insertion, in minimally invasive interventions, into respiratory organs lying deep inside the body, such as the segmental bronchi.

It will be appreciated that the presence of an external cuff will be advantageous in any case, said cuff being provided on the outside of the tube body, between the distal end and the proximal end thereof.

According to the invention, an optionally provided cuff can also be inflated in a manner known per se via a pressure conduit which is connected fixedly to the tube body and/or is integrated therein or thereon. As will be explained in more detail below, the inflation can also be controlled via an electronic control system on which, preferably, images from the inside of the body are then also displayed and/or with which the steering of the tube that is to be positioned is facilitated or permitted. It will be noted that, although it would in principle be possible to acquire images at the cuff itself or also at any desired area of the jacket of the tube body and to send them outside the body in order to achieve improved monitoring and viewing, the cuff generally provides the anesthetist or surgeon or other medical personnel with a view from outside, and other means, for example acoustic controls, exist for control of a suitable cuff position and cuff underpressure.

It will be noted that, for the endotracheal tube of the present invention too, the cuff preferably comes to lie below the vocal cords, that is to say in the trachea. With the present invention too, it should preferably be ensured that it does not come to lie in the vocal cord plane and thus damage the possibly sensitive vocal cords.

In this regard, particular mention will be made of the possibility of providing an elongate low-pressure cuff, which is advantageous when the position of the cuff cannot be entirely predicted at the start of the operation because the intervention depth varies with the progress of the operation and/or is not yet entirely clear at the start of an intervention. It will also be noted that several cuffs can be coupled together and/or, as in thoracic surgery, double-lumen tubes with two cuffs can be used.

Whereas in conventional intubation, with viewing through a laryngoscope, the tube is typically advanced until the cuff lies safely behind the vocal cords and has thus disappeared from visual range, it is possible in the present invention, in the preferred variant with fiber-optic intubation, to verify the correct position of the tube preferably by the fact that the end of the tube is placed above the tracheal bifurcation, i.e. the first branching of the trachea into the left and right main bronchi. If the passage of the cuff through the vocal cord plane cannot be observed by laryngoscope, for example because fiber-optic intubation with steering according to the invention is being used precisely for lack of a laryngoscopic view, a correct position of the cuff, that is to say the placement of the cuff below the vocal cords, can be achieved by distance markings on the outside of the fibus, for example in the centimeter or double centimeter range.

It can thus be ensured that the cuff comes to lie safely below the vocal cord plane, by determining the fibus depth starting from the row of teeth. If it is unclear how deep the interventional aid is to be inserted for an intervention and/or if the depth of insertion varies during the intervention, a cuff can be provided which is either longer than is necessary at least for sealing an individual fixed insertion position and/or which can be inflated segment by segment. As has been explained, it is particularly preferable that the functional aids comprise conducting means for transmitting images from the inside of the body, said means being connected fixedly to the wall while leaving the lumen that permits ventilation.

These conducting means for transmitting images will on the one hand comprise means of illumination, since the transmission of images from the inside of the body is possible only with illumination. Reference is made to the possibility of using a light-conducting fiber for illuminating at least one location in the inside of the body. Several light-conducting fibers can preferably be arranged in or on the tube body in order to ensure sufficient illumination even when the light outlet point of an individual fiber is concealed, as can happen as a result of mucus or blood in the inside of the body. Moreover, the conducting means will typically comprise at least one image-conducting fiber, such as a glass fiber or synthetic fiber. These can easily be integrated into the tube jacket. As regards the refraction index differences required for light-conducting glass fibers in relation to ambient media, conventional techniques known per se can be used to introduce the glass fibers into the tube jacket. Alternatively and/or in addition to image-conducting fibers, it is possible to provide sensor signal lines in order to convey analog or digital sensor signals, in particular for imaging systems, out from the inside of the body. Such a sensor can be formed as an array of light-sensitive elements or as an ultrasound sensor, and it should be noted that the resolution necessary for the respective sensor and attainable thereby is to be adapted to the respective purpose of the intervention and to the respective requirements.

In a particularly preferred variant, a multiplicity of image entry openings or image capture openings are distributed at the proximal end of the tube. The distribution of image entry openings, whether in the form of several arrays of light-sensitive elements or in the form of image-conducting glass fibers spaced apart from one another, has the advantage that, as long as the view via several image conductors (sensors, glass fibers, etc.) is unimpeded, a stereoscopic image can be made available to the person using the interventional aid and/or to other persons who are participating in a medical intervention. For sake of completeness, it will be mentioned that the image can be presented on monitors, purely optically by suitable lenses, prisms, etc., or by means of stereoscopic glasses with miniaturized monitors. If, as may happen in interventions, the proximal image entry openings or image capture openings become partially obstructed, it is still possible, if appropriate without stereoscopic viewing, to transmit an image from the inside of the body, such that an intervention no longer has to be interrupted because of mucus, for example, collecting in front of the image entry openings. This is particularly advantageous since replacement of the tube also leads to a temporary interruption in ventilation. Therefore, in a preferred variant, the image entry openings are provided for stereoscopic purposes and/or redundancy. It will be noted that several glass fibers can be provided for purely optical viewing, for example, while a monitor can be attached to a third image channel to allow the medical intervention to be displayed on large screens, for example for training students in university hospitals.

In a preferred variant in which image-conducting means are present, it is possible to provide anti-misting means and/or anti-adhesion means at the image entry openings or, if the image-conducting means comprise them, at the sensors. These means can be obtained by a coating or the like, formed for example with a biocompatible fluorinated synthetic compound. Such means are advantageous because it is not only possible for blood, mucus or other material to conceal the entry opening or outlet opening of the corresponding image-conducting means, but the humidity of the respiratory air can already cause misting, which could be extremely inconvenient if the tube during intubation is cooler than the inside of the body already surrounding it and/or it permanently remains slightly colder than its environment as a result of cooler respiratory gas.

Alternatively and/or in addition to anti-misting means and/or anti-adhesion means, it is possible to provide other means for keeping the optical system clean, for example for keeping the optical system clean by means of continuous or intermittent insufflation of a suitable gas for flushing clear the image entry openings. It will be noted that, for this purpose, additional channels can also be provided in the wall of the endotracheal tube and/or the image-conducting means can be routed such that air or other fluid can circulate around them and flush them.

In a particularly preferred variant, the tube body will be open at the long side to permit ventilation. However, it would be possible to permit the ventilation only via lateral openings in the tube wall before the proximal end, if a minimally invasive surgical instrument with a proximal thickened area is to be inserted.

It has already been mentioned that the interventional aid of the present invention can be used not only for intubation of the kind required in anesthesia and the like, but also for intubation deep into the main bronchi, preferably into the secondary and/or segmental bronchi. It will be appreciated that this is associated with an increase in length compared to conventional intubation tubes, such that lengths of greater than 50 cm, preferably of 60 cm or more, can be provided for adult patients of normal size.

The preferred variant will not only have a single Murphy eye for permitting ventilation even when the proximal end of the tube is closed, but also, as has been mentioned, a plurality of lateral openings which are expediently arranged at the proximal side (inside the body) behind the cuff. A lateral opening before the cuff could result in penetration of saliva into the tube, which is extremely undesirable for obvious reasons. It will be noted that, in a preferred variant in which steering means and also image-conducting means run through the tube wall and in which the steering means are provided for controlling the tube in more than one plane, such that three of four control wires run through the tube wall, it will be preferable to group the conducting means guided through or along the tube wall such that the lateral (secondary) openings, which are also intended to permit ventilation of the lung areas not controlled by the intubation tube, can be positioned without disturbing the functional aids. Alternatively and/or in addition, provision can be made to turn the tube about its longitudinal axis. During the insertion phase in particular, in which the tube is securely gripped anyway, this is a simple and convenient method of orientation, especially in combination with a steering system comprising two tensioning filaments. As has already been mentioned, the lateral openings increase the flexibility of the interventional aid in a positive way, especially if several such openings are arranged along the tube wall. This is regarded as advantageous, specifically in view of the aim that the interventional aid is to be easier to position.

It will further be noted that, in the typically preferred steering of the tube end in two planes, it is not absolutely necessary for four control wires to be arranged on mutually orthogonal diameters along the cross section of the tube wall. It is true that the actuation of such an orthogonal arrangement of control wires is more intuitive and helps the physician position the tube under purely mechanical control. However, it is alternatively possible to provide only three wires, for example, especially if the tube wall is densely occupied by functional elements. In order then to provide the intuitive leftward/rightward/upward/downward control of the end of the tube, it is possible, in a particularly preferred variant, to use an electronic control system which converts the desired movement signals into corresponding tensional forces on then only three tensioning wires. Such an arrangement may be preferable in interventional aids that have to be made very thin because they are intended to be inserted deep into the lung, have to be flexible and/or are to be used on younger patients, particularly children.

It will be noted that the tube wall can be designed with axial through-holes, that is to say small guide channels. This is particularly preferable per se and can be easily accomplished in the production of the tube body. In such a case, care will typically have to be taken to ensure that the channels provided around the functional aids on the tube wall or image-conducting means on the tube wall remain at least thick enough toward the outside, that is to say the body contact side, so that no damage can be caused in the insertion phase, e.g. by kinking or piercing; the latter would have be to be expected, particularly in emergency cases, on teeth, etc. On the lumen side, by contrast, a thickness should be provided that is sufficient to withstand the forces which may be occasioned by inserted instruments for a minimally invasive intervention. It will be appreciated that comparatively small forces occur in both cases, which permits a correspondingly small residual wall thickness on condition that no thermally acting minimally invasive devices or the like are pushed through that could cause the tube wall to melt or could bring intensive laser beams or the like to the tube wall. Consequently, the dimensioning of the channel can and typically will be based purely on considerations relating to the stability of the tube. This makes it possible in particular, and in a preferred variant, to integrate a separate suction channel directly into the tube wall via which an intervention site can be suctioned from the outside and/or suctioning is also possible at other locations, for example at a distance around the intervention site, from the outside.

In a particularly preferred variant, the interventional aid will comprise, on the one hand, a proximal part that is inserted into the patient and must therefore have intensive contact and, on the other hand, an operating end or control end that is free of contact with the patient, which parts are preferably separable from each other. This separation means that, for the proximal part in patient contact, it is possible to provide inexpensive disposable components, which is possible if the orientation means, image-conducting means and the like can be embodied by integration or application of inexpensive fibers, wires, cords and the like. Where this is not possible, or no longer completely possible, for example because expensive sensor elements are to be used instead of image-conducting glass fibers, it is alternatively preferably possible to carry out at least simple disinfection and sterilization.

It will also be noted that, if appropriate, further functional aids can be provided on the interventional aid, particularly at the proximal end thereof, for example electrodes for electrostimulation, stimulus measurements, electrosurgery and the like.

A part free of contact with the patient will be formed as a command part and/or operating part, which is designed in particular with attachments for suction means and/or ventilation means, monitors for gas (partial) pressures and/or image signals received from inside the body and/or can be connected to these and preferably also comprises the actuating means for controlling the tube movement, which can electronically and/or mechanically receive the desired direction of movement or orientation. It will be noted that, in a particularly preferred variant, a trocar seal can be provided for the distal part.

The invention is described below, merely by way of example, with reference to the drawing, in which:

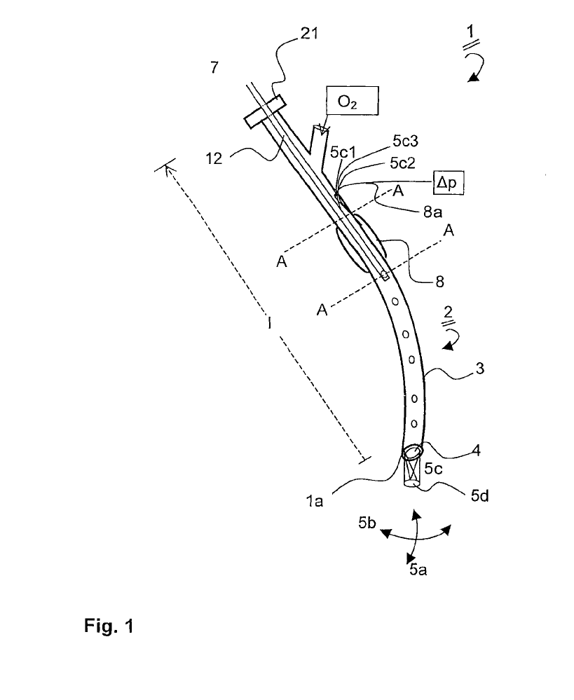

FIG. 1 shows an interventional aid for medical interventions;

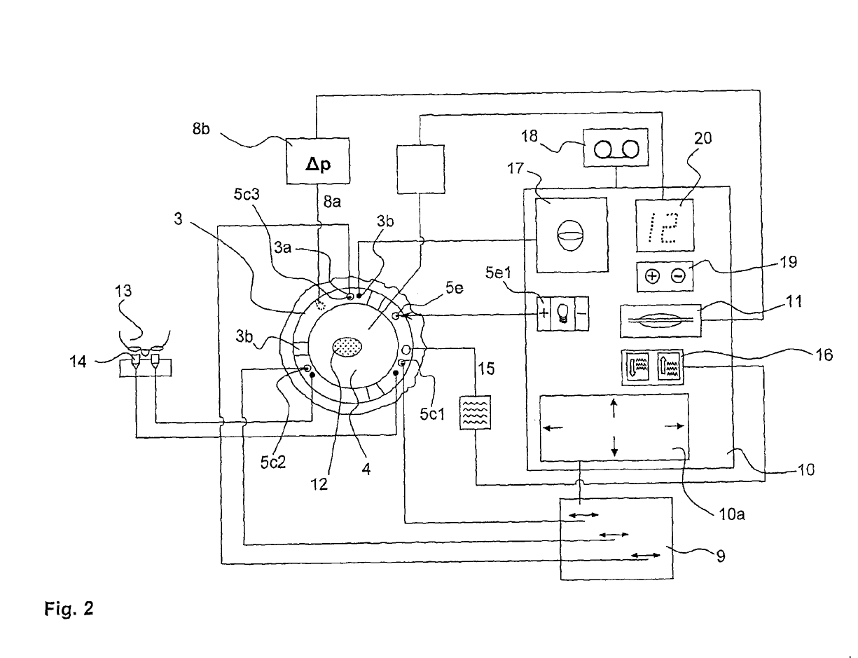

FIG. 2 shows the interventional aid from FIG. 1 in cross section along A-A in FIG. 1, with a control device and an eyepiece for a surgeon.

According to FIG. 1, an interventional aid 1 designated overall by reference number 1 and used for medical interventions comprises a tube body 2 with a tube jacket 3 which is suitable for intubation and which, while leaving a lumen 4 to permit ventilation, is provided with a functional aid 5 for performing an interventional function, wherein the functional aid 5 is designed with an orientation means 6 for orienting the tube body during a medical intervention.

The interventional aid 1 is a tube for endotracheal intubation which is made from flexible biocompatible material and which, in order to make the intubation easier, is beveled at the proximal end 1a in the usual way, as is preferably possible but not absolutely necessary, and the edges of the tube body at the proximal end are rounded in order to avoid injury.

The tube body 2 is formed with such an external diameter that it can be inserted into the trachea of a patient who is to be treated and who is assumed here to be under anesthetic, although this is not necessary in emergency cases. The tube body 2 is in particular compatible with the usual lubricants and the like that facilitate intubation. The tube jacket 3 has a thickness which, even taking into account the functional aids arranged therein and extending through channels 3a, 3b, etc., gives it a sufficient stability against pressure from outside and in particular also against kinking when a pressure is applied at the distal end 7 for pushing it forward.

The tube body 3 has a length 1 which here allows the proximal end of the tube body to be pushed from the pharynx into the segmental bronchi. Around the tube body 3 of the interventional aid 1 according to the invention designed thus as an endotracheal tube 1, there is a cuff 8 which lies below the vocal cords, after the proximal end 1a of the tube body 3 has been put in place, and which is connected in the usual way via a conduit 8a in the tube wall, so as to be able to be supplied with pressurized fluid from a pressure source 8b (FIG. 2). The compressed air is supplied under the control of an intervention control system 10 by which a pressure for inflating the cuff can be predefined, or by the control of which air or another pressurized fluid can be removed from the cuff when it is necessary to reposition the endotracheal tube during a medical intervention and/or remove the endotracheal tube 1 from the cuff. It will be noted here that the control system 10 is provided with a command 11 for the cuff pump 8b, although this does not necessarily have to be the case, and it would be entirely possible to actuate the cuff manually, although this is less preferable.

As regards the optimal cuff pressure, it will be noted that this can be adjusted preferably automatically to the optimal cuff pressure by which is understood the pressure that is needed to avoid leakage of respiratory gas or entry of liquids, such as saliva or gastric juice, at given ventilation pressures. A leakage can be determined, particularly in anesthesia devices, by measuring a difference between inhaled and exhaled gas. Physicians are also aware that sufficient leaktightness is detectable by the absence of a “bubbling sound”.

The tube body 3 is further provided with openings 3b which extend radially outward through the wall and which, in the case of deep intubation as far as one of the secondary segmental bronchi, permit ventilation of the lung areas lying upstream of the proximal tube body end 3 by secondary ventilation. The openings 3b can be arranged at any desired location of the tube body 3, provided they are deeper in the inside of the body than the cuff 8 and provided they do not pass through any functional aids and the like extending axially in the tube wall. The openings generally extend radially outward, it being noted here that an inclination with respect to the axis is preferably possible in order to avoid penetration of a minimally invasive instrument into an opening if the instrument is displaced in the tube.

The number and size of the ventilation holes 3b are such as to readily ensure that the lungs are supplied with respiratory air, if appropriate respiratory air to which anesthetic gases have been added, such as nitrous oxide, etc.

The size of the lumen 4 is chosen such that ventilation is possible even when one or more surgical instruments 12 are inserted, such as forceps for removing foreign bodies from the bronchi, for performing biopsies, for removing endogenous substances, or scalpels, electronic scalpels, scissors, etc. It will also be noted that, in contrast to what is shown, for interventions to be performed deep within the respiratory organs, the tube body 3 does not necessarily have to be cylindrical and designed with a preliminary curvature, and instead it may also be possible, for example, for the tube body 3 to have a stepped design in such a way as to allow the interventional aid 1 to be pushed deep into the ramifications of the bronchi. In such a case, a stepped design can be provided in such a way that the lumen still remaining at the proximal end is itself barely sufficient for the overall ventilation, provided that corresponding side holes 3b and the associated distally larger lumen 4 then ensure that the ventilation and oxygen supply of the patient is not impaired.

The functional aid 5 comprises several tensioning wires which engage on the proximal end of the tube body 3 in order to orient the latter in two planes, indicated by arrows 5a, 5b in FIG. 1. For this purpose, in the illustrative embodiment shown in FIG. 2, channels 5c1, 5c2, 5c3 are distributed uniformly in the tube 3 and equidistantly across the tube diameter.

In principle, it would be possible to provide a separate pair of wires for leftward/rightward and upward/downward movement, that is to say in the first instance corresponding to the arrow 5b and in the second instance corresponding to the arrow 5a, which in total would require four tensioning wires extending in the tube wall. In the present case, an arrangement is shown in which the desired movements are provided using only three tensioning wires 5c1, 5c2, 5c3, which each extend in a dedicated channel and are controllable via the control system 10, with a command panel 10a provided thereon and formed for example as a touchpad or as a rocker switch with dual rocking.

For example, an upward movement in FIG. 2 can be effected by pulling on the wire 5c3, while a downward movement can be effected by pulling with equal strength on the wires 5c1 and 5c2. Conversion of the intuitively more easily understandable leftward/rightward/upward/downward movement to three tensioning wires is effected electronically in a manner known per se, such that the medical practitioner, who has to place the interventional aid 1 in position, is not distracted by complex considerations, for example over orientations.

The tensioning wires 5 in their respective channels can be seen in conjunction with the control system 10a as the orientation means. Without compromising the disclosure offered to the technician, it will be noted, purely for reasons relating to patent protection, that different parts can be assigned to the orientation means. For example, the control system 10 described here by way of example and as being advantageous, with the command means 10a for the tensioning wires 5c1, 5c2, 5c3, is not essential, and instead it is also possible to use mechanical means in a manner known per se. A device is then preferred that can be actuated with one hand, as is already provided in the prior art.

The functional aids further comprise image-conducting means which in the present case are embodied as three glass fibers that extend parallel to the tensioning means and that are positioned at the proximal end of the tube body 3 such that one and the same image area is observed, as is indicated schematically by the image area 5d in FIG. 1. The intervention is also supported by transmission of an image from inside the body, such that the image-conducting means can also be interpreted as functional aids within the meaning of the present invention. Moreover, a glass fiber 5e (FIG. 2) is provided via which the site can be illuminated with a brightness level that can be adjusted by the control system 10, indicated by the adjustment means 5e1.

The arrangement of three image-conducting fibers in the tube body 3, in particular glass fibers, allows a surgeon to be provided with a stereoscopic image of the site in the inside of the body, which is of great advantage for medical interventions such as biopsies or surgical interventions. Moreover, images from the inside of the body can also be displayed via the third glass fiber on the control system 10 to a person assisting the surgeon, for example the anesthetist. It will be appreciated that the binocular eyepiece for the surgeon, which is designated as 14 in FIG. 2, does not necessarily have to work purely optically, and instead a conversion to electrical signals is possible and the surgeon then wears electronic glasses, which show a stereoscopic image, or looks at a monitor that permits stereoscopic viewing. The latter in particular has the advantage that, in the event of one of the image-conducting fibers being blocked, it is always possible to change to the remaining ones in order to obtain a stereoscopic view, which makes positioning of the tube easier, and further individuals are able to view the site stereoscopically.

Other functional aids which are provided on the interventional aid 1 in the illustrative embodiment shown but which, as will be evident to a person skilled in the art, do not necessarily have to be present, are a flushing means 15 for flushing the distal end of the interventional aid 1, and FIG. 2 illustrates, very much by way of example only, a control system in the form of two push buttons 16 for delivery of flushing media and for removal of flushing media.

It will further be noted that, in addition to a monitor 17 for displaying an image recorded at the proximal end, the control system 10 also comprises an attachment for recording all the commands and all the signals received from the inside of the body, this recording means being indicated in the present case as a tape drive 18, although typically the data can be digitized and stored on a central computer or the like, and a control system for the patient's respiration, for example an oxygen increase or decrease can be controlled and can be indicated to an anesthetist or other physician on a display 20 via corresponding keys 19 and certain parameters. It will also be understood that the control system 10 can if appropriate be coupled to other monitors and the like, in order to indicate at a combined site parameters such as pulse rate, blood pressure, etc., such that a physician monitoring an intervention can view all the critical variables simultaneously. It will also be clear that critical states of individual variables can be indicated acoustically.

The interventional aid 1 is, for example, used in the following way:

First, a patient on whom the interventional aid is to be used is anesthetized as required. The interventional aid 1 is then pushed into the patient's trachea, preferably but not necessarily using a conventional laryngoscope for the anterior insertion area. In addition to the fact that, in difficult intubations, a laryngoscopic view of the laryngeal area and/or of the vocal cords is not achievable, it will further be noted that in such situations the endotracheal tube of the present invention can then advantageously be used without additional aids. An advance movement into the esophagus can be immediately detected and corrected by observing the monitor 17, such that there is no risk of incorrect intubation. In order then to position the interventional aid 1 at a desired site deep within the body, for example at the segmental bronchi, an insertion pressure is applied from the distal end, with orientation of the end of the tube body by means of the three tensioning wires 5c1, 5c2, 5c3, which are tensioned or released by the actuator 10a of the control system 10, and the command means 9 is correspondingly controlled until a desired site for the intervention is reached. For completeness, it will be noted that in this process the cuff can be inflated and deflated in a manner known per se.

Even if the advance of the tube takes longer than is desired, the ventilation of the patient is ensured at all times, since the integration of the control wires in the wall leaves the lumen 4 free for viewing the position of the endotracheal tube during intubation.

It is also the possible for surgical instruments, if so required, to be inserted through the trocar 21 at the distal end 7 of the tube body 3. Since these instruments can be made so thin, according to the prior art, that the lumen is not significantly impeded in terms of the ventilation, ventilation continues to be guaranteed during this period. The site can be permanently observed by images being conveyed via the glass fibers 5c1, 5c2, 5c3, which can be routed laterally out of the tube body. A permanent supply of oxygen to the patient is also ensured via the oxygen or respiratory gas supply line ending in the lumen 4. Even in cases when large foreign bodies have to be removed from the bronchi, which foreign bodies at least substantially block the proximal end of the tube when they have been gripped by microsurgical tools, the lateral openings 3b provided in the tube jacket 3 ensure that ventilation continues until the endotracheal tube 1 of the present invention has been removed from the patient.

If the tube needs to be repositioned during the medical intervention, for example in order to collect tissue samples from different locations in a biopsy or in order to perform an intervention along a considerable stretch of an airway, it is possible, by orienting the tube end by actuation of the command panel 10a, to obtain a corresponding movement without inserting additional tools. This reduces the duration of the intervention not inconsiderably, such that a patient is able to undergo a shorter procedure. Moreover, a satisfactory supply of oxygen is permanently ensured. It will be noted that in cases where the endotracheal tube is used for operations or the like in which it has to be regularly reoriented and/or repositioned by the surgeon, it is possible to provide both the anesthetist and also the surgeon with different and, if appropriate hierarchically or temporarily dominating, command panels 10a and/or to provide only one command panel which dominates hierarchically, temporarily or selectively, or to separate the command means entirely from the control system 10 in order to allow the anesthetist a better monitoring of the patient from the orientation once it has first been positioned.

In contrast to what has been described above, it is not absolutely necessary to use just one continuous lumen. It would be possible also to subdivide the lumen and/or provide a guide device for guiding several endoscopic microsurgical instruments, for example to ensure that endoscopic scissors do not get caught on forceps in the interior or the working lumen. It must also be stressed that other endoscopic instruments can also be used in the fibus, for example a laser device or laser beam emission device, an electrocoagulation tip, foreign-body traps such as Dormia baskets, balloon catheters for dilation and/or bronchial clearance (Fogarty catheter), catheters for introduction of radioactive isotopes and/or a device for inserting stents in tumor diseases.

1. An interventional aid for medical interventions, comprising:
a tube body with a tube jacket which permits intubation and which, while leaving a lumen to permit ventilation, includes a functional aid for performing an interventional function, wherein the functional aid includes an orientation means for orienting the tube body during the medical intervention.

2. The interventional aid as claimed in claim 1, wherein it is designed for at least one medical intervention from the group comprising bronchial lavage, bronchoscopy, biopsy, anesthesia, intensive care, intubation, aspiration and/or for training in such an intervention.

3. The interventional aid as claimed in claim 1, designed for a minimally invasive intervention, preferably on or beyond the main bronchi, in particular in the segmental bronchi.

4. The interventional aid as claimed in claim 1, wherein an external cuff is provided on the tube body, between the distal end and the proximal end of the latter.

5. The interventional aid as claimed claim 4, wherein the cuff can be inflated via a pressure conduit which is connected fixedly to the tube body and/or is integrated therein or thereon.

6. The interventional aid as claimed in claim 4, wherein a segmented cuff is provided and/or a cuff that is longer than is necessary for at least sealing in an insertion position.

7. The interventional aid as claimed in claim 1, wherein the functional aids further comprise conducting means for transmitting images from the inside of the body, said means being connected fixedly to the wall while leaving the lumen that permits ventilation.

8. The interventional aid as claimed in claim 7, wherein the conducting means comprise a means of illumination, in particular a light-conducting fiber for illuminating at least one location in the inside of the body.

9. The interventional aid as claimed in claim 7, wherein the conducting means comprise at least one image-conducting fiber, preferably a glass fiber.

10. The interventional aid as claimed in claim 7, wherein the conducting means comprise at least one sensor signal line in order to convey analog and/or digital sensor signals, in particular for imaging systems, out from the inside of the body.

11. The interventional aid as claimed in claim 10, wherein at least one sensor is formed as an ultrasound sensor and/or as an array of light-sensitive elements.

12. The interventional aid as claimed in claim 7, wherein a multiplicity of image entry or image capture openings are distributed at the proximal end of the tube.

13. The interventional aid as claimed in claim 12, wherein the image entry openings are provided for stereoscopy and/or redundancy and/or additional detection purposes.

14. The interventional aid as claimed in claim 7, wherein anti-misting means and/or anti-adhesion means are provided on or at the image entry openings.

15. The interventional aid as claimed in claim 1, wherein the tube body is open at the ends to permit ventilation.

16. The interventional aid as claimed in claim 1, wherein the tube body is dimensioned for intubation at least as far as the main bronchi, preferably as far as the secondary bronchi, in particular with a length of greater than 50 cm, preferably of 60 cm or more.

17. The interventional aid as claimed in claim 1, wherein at least one Murphy eye, preferably a plurality of lateral openings are arranged in the tube wall at the proximal side (inside the body) from the cuff.

18. The interventional aid as claimed in claim 1, wherein the lateral openings in the tube wall are dimensioned to permit ventilation.

19. The interventional aid as claimed in claim 1, wherein the lumen is dimensioned such that minimally invasive elements can be pushed through it, preferably while allowing sufficient ventilation in the moderate to long term.

20. The interventional aid as claimed in claim 1, wherein the fixed connection between tube wall and at least one functional aid conducting means further elements guided along the tube wall is effected by adhesion and/or by forming them in or on the wall.

21. The interventional aid as claimed in claim 1, wherein the orientation means for controlling a tube movement in the inside of the body, in particular during insertion, is preferably provided with steering means guided on and/or in the tube wall.

22. The interventional aid as claimed in claim 21, wherein the orientation means are designed to permit control in two orthogonal planes.

23. The interventional aid as claimed in claim 1, wherein a separate suction channel is provided.

24. The interventional aid as claimed in claim 1, comprising at least one proximal part for contact with the patient and at least one part free of contact with the patient, which parts are separable from each other.

25. The interventional aid as claimed in claim 1, wherein the part free of contact with the patient is formed as a command part and/or operating part, which is designed in particular with attachments for suction means and/or ventilation means, monitors for gas (partial) pressures and/or image signals received from inside the body and/or can be connected to these and/or comprises control means for the tube movement.

26. The interventional aid as claimed in claim 1, wherein a trocar seal is provided for the proximal part.
